(19) United States
(12) Patent Application Publication
(10) Pub. No.: US 2016/0130545 A1
(43) Pub. Date: May 12, 2016

(54) BIOLOGICAL LOAD FOR BIOREACTOR

(71) Applicant: Mikhail PUKEMO, Moscow (RU)

(72) Inventor: Mikhail PUKEMO, Moscow (RU)

(21) Appl. No.: 14/933,284

(22) Filed: Nov. 5, 2015

(30) Foreign Application Priority Data
Nov. 7, 2014 (RU) 2014144740

(51) Int. Cl. C12M 1/12

(52) U.S. Cl. CPC C12M 25/10

(57) ABSTRACT

The present invention relates to the field of wastewater treatment witch deals with the biological load for bioreactors. The biological load for the bioreactor having a cylindrical shaped housing with an inner surface and an outer surface, said housing comprises of an inner layer having a diameter placed at said inner surface made of a low-density polyethylene with a polymorphic structure; a middle layer to form a supporting frame of the biological load having a gradient porous transition into an outer layer; said outer layer formed by a canvas of polymer filaments made of polypropylene fiber, said canvas formed as a plurality of rowlock arch shaped distributed over said outer surface. The canvas can be used as a component of deep biochemical and biological treatment for domestic wastewater.

### FIELD OF THE INVENTION

The present invention in general relates to the field of wastewater treatment, and in particular, it deals with the biological load for bioreactors which contain polymer filaments formed into a canvas with many uniformly distributed loops over its surroundings.

### BACKGROUND OF THE INVENTION

The prior art discloses the biological load for bioreactors which contains polymer filaments with Capron line and stainless steel wire as a base. The prior arts have too many drawbacks such as short service life. Corrosion which happens in bioload construction and the design of the bioload construction are the main reasons for the short service life in the prior arts.

In prior arts problem caused by low quality biological load and additionally with the absence of a rigid frame which leads handling biological load with difficulty.

There is a need for a rigid biological load for bioreactors which has a specific construction that is corrosion resistance and can handle biological load.

### SUMMARY OF THE INVENTION

The present invention is a biological load for biological filter for wastewater treatment. The invention is a type of bioload, which contains uniformly distributed loops of polymer filaments formed over the entire surface of a canvas. The canvas is adapted to be placed inside a cylindrical space with the loops appearing on the outside of the cylinder. This special canvas is used in biochemical and biological treatment of domestic wastewater and it is similar in composition to them.

The biological load—the base for the biological filter, which is a part of the bioreactor, in which wastewater is filtered through the biological load, is coated by biological film (biofilm) formed by colonies of microorganisms.

The main purpose of the present invention is to propose a biological load for bioreactors which will at minimum offset, to solve the prior arts drawbacks and also it provides an improvement on the technical specifications of the biological load, namely: an improvement in the ratio between the surface area and the volume of the biological load, an increase in the corrosion resistance, an increase in the structural integrity of the frame of the biological load, and an improvement in the practicability of the assembly and maintenance of the biological load.

For such an achievement, the biological load for bioreactors has an additional inner layer made of low-density polyethylene with a porous structure.

dUE to these advantageous characteristics, it is possible to aerate the bulk biomass to the layers both from inside and outside with aeration nozzles or hydro-aerators ejecting pressurized air into the water. Hydro-aerators enable airlifting with additional bubble grinding through the porous walls of the base (the middle layer) and removal of biomass from the biological load. Additionally, with these characteristics, the inner layer promotes the formation and retention of a multilayer biofilm and the development of aerobic and anaerobic biological forms within the space of this layer.

The biological load for bioreactors also have a middle layer which forms the supporting frame and has a gradient porous transition into an outer layer formed from a canvas of polymeric filaments.

It is possible to achieve the desired strength in the structure of the present invention by increasing the density of the layer and by decreasing, to a certain extent, the permeability of the flow after passing the outer layer, to disperse large amount of air bubbles and to create a base for the formation of the next layer.

The outer layer of the bioload is formed from a canvas of polymeric filaments, made from polypropylene fiber.

This bioload allows for an increase in the apparent viscosity of the water flow, which helps to capture more of suspended nutrients.

In the preferred embodiment of the present invention, the inner layer of the bioload has a porosity of up to 71%. This provides an optimal condition for the aeration of the main biomass.

In the preferred embodiment of the present invention, the pores of the inner layer are uniform in size, and preferably are 1.5 mm is size. This size provides an optimum condition for the aeration of the main biomass.

In the preferred embodiment of the present invention, the surface area of the space between the inner and outer layers is not less than 750 m²/m³. This provides an optimal condition for the aeration of the main biomass.

In another embodiment of the present invention, the middle layer possesses a diagonal thickness ranging from 0.5 to 2% of the exterior diameter of the inner layer to provide certain rigidity to the frame.

In another embodiment of the present invention, the middle layer has a varying porous layer, having a smooth transition into the third outer layer. This provides an optimal condition for the dispersion of large air bubbles.

In another embodiment of the present invention, the middle layer is created possessing a ratio between the sizes of the pore channels of the inner and outer boundary equal to two. This allows for having different types of middle layer.

In another embodiment of the present invention, the outer layer has a specific surface area of no less than 1220 m²/m³. This makes it possible to build a multilayer biofilm with a large amount of biomass bridges and to capture a greater amount of suspended nutrients.

In another embodiment of the present invention, the outer layer possesses a density no less than 95%. This provides an optimal condition for biomass growth.

Other objects, features, and advantages of the present invention will be readily appreciated from the following description. The description makes reference to the accompanying drawings, which are provided for illustration of the preferred embodiment. However, such embodiments do not represent the full scope of the invention.

### BRIEF DESCRIPTION OF THE DRAWINGS

Embodiments herein will hereinafter be described in conjunction with the appended drawings provided to illustrate and not to limit the scope of the claims, wherein like designations denote like elements, and in which:

FIG. 1 shows a two dimensional view of the biological load for bioreactors according to the present invention

FIG. 2 shows a schematic cut-away view through the line A-A in FIG. 1;

FIG. 3 shows microscopic view of the polymeric filament used in the outer layer of the present invention;

FIG. 4 shows a typical wastewater treatment with the biological load of the present invention;

FIG. 5 shows a schematic diagram of a wastewater treatment plant with the present invention;

FIG. 6 shows the operational workflow of the present invention in a treatment plant; and

FIG. 7 is preferred physical and mechanical characteristics of the present invention for a biological load of a bioreactor.

### DETAILED DESCRIPTION OF PREFERRED EMBODIMENTS

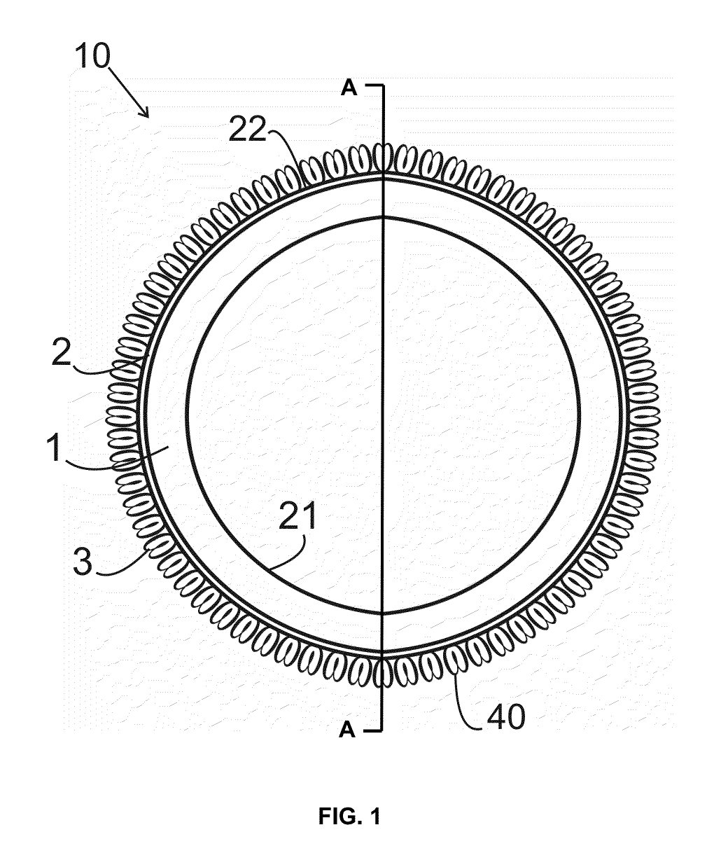

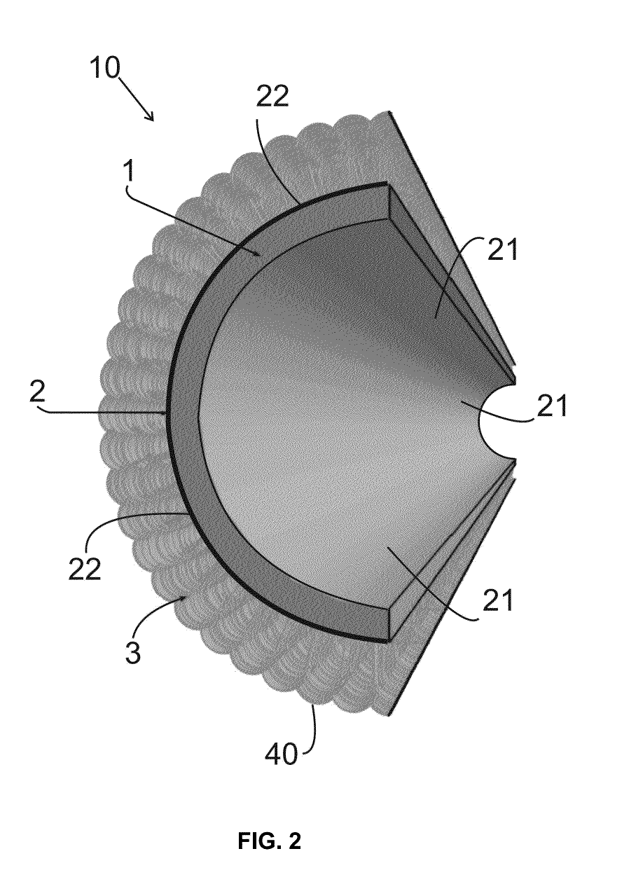

FIGS. 1 and 2 schematically show the appearance of the biological load 10 for bioreactors according to the present invention. As shown in FIGS. 1 and 2, the biological load 10 for bioreactors includes three layers: inner layer 1, middle layer 2 and outer layer 3.

Again as shown in FIGS. 1 and 2, a biological load 10 for a bioreactor having a cylindrical shaped housing with an inner surface 21 and an outer surface 22, the housing comprises of an inner layer 1 having a diameter and it placed at the inner surface 21 made of a low-density polyethylene with a polymorphic structure; a middle layer 2 to form a supporting frame of the biological load 10 having a gradient porous transition into an outer layer 3; and the outer layer 3 formed by a canvas of polymer filaments 40 made of polypropylene fiber, the canvas formed as a plurality of rowlock arch shaped 40 distributed over the outer surface 22.

The inner layer 1 promotes the formation and retention of a multilayer biofilm and the development of aerobic and anaerobic biological forms within the space of the layer.

The inner layer 1 is made of high-density polyethylene and has a porous polymorphic structure. The middle layer 2 forms the supporting frame of the structure and has a gradient porous transition into the outer layer.

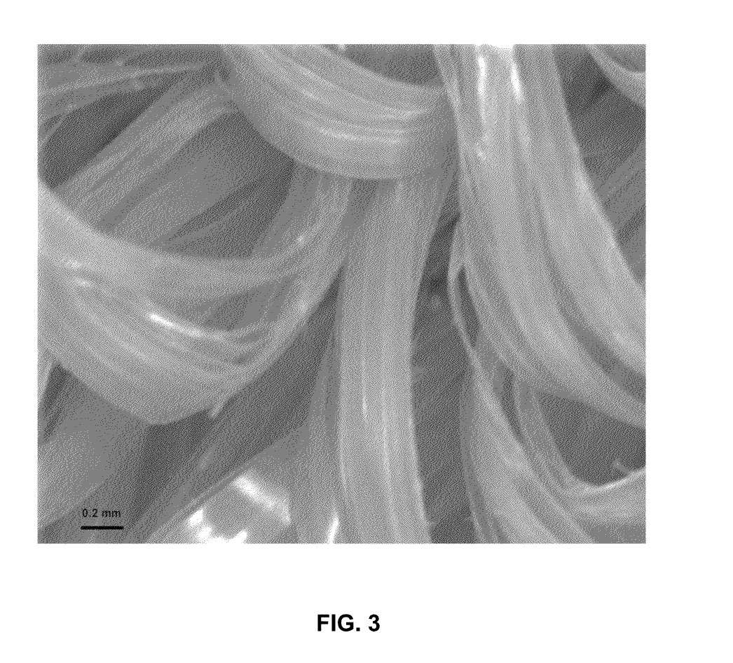

The outer layer 3 is formed from a canvas of polymer filaments as shown in FIGS. 1-3. The polymer filaments are formed into a canvas of pluralities of uniformly distributed loops 40 over its surrounding. The canvass is adapted for placement inside a cylindrical space with the loops 40 appearing on the outside of the cylinder. The outer layer 3 is made of polypropylene fiber. The design and construction of the outer layer 3 helps to capture more of suspended nutrients in the waste water treatment.

In one embodiment of the present invention, the inner layer 1 is formed with a porosity of up to 71%, with linear values for the size of the pores up to 1.5 mm, and with the surface area of the space between the inner 1 and outer layers 3 is at least 750 m²/m³.

The middle layer 2 is formed with a diagonal thickness ranging from 0.5 to 2% of the exterior diameter of the inner layer, with a gradient porous transition into a third outer layer 3, and having a ratio between the sizes of the pore channels of the inner and outer boundary equal to two.

The outer layer 3 has a specific surface area of at least 1220 m²/m³ and a density of at least 95%.

### IMPLEMENTATION OF THE PRESENT INVENTION

The implementation of biological load for bioreactors as an example is shown which is describe one embodiment of the present invention and does not limit the application of the present invention. The usage of bio-filter in an Alta Air Master treatment plant is provided as an example of the utility model.

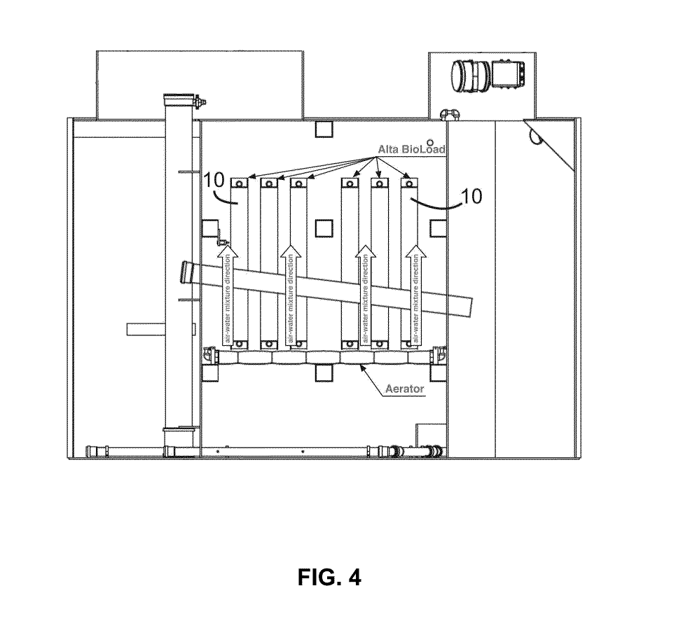

FIG. 4 shows a typical wastewater treatment with the biological load of the present invention. As shown in FIG. 4, the biological load 10 is located in the tank which an aerator mixes air with water. The mixture of air and water contact with inner and outer surface of the biological load during the circulation. The loops designed in the outer surface of the biological load 10 helps to capture more of suspended nutrients in the waste water treatment. Because of the design of the outer surface of the biological load 10, the colonies of microorganisms, which are the basis of the bio-filter, begin to multiply and colonize the space formed by the loops and canvas of the biological load 10.

During the circulation, because of the specific design of the biological load, the maximum contact between the biological load and air-water mixture happens. By the time, the colonies of microorganisms grow in the spaces between the loops in the outer surface and by circulation connect to each other. The heavy colonies fall down the tank.

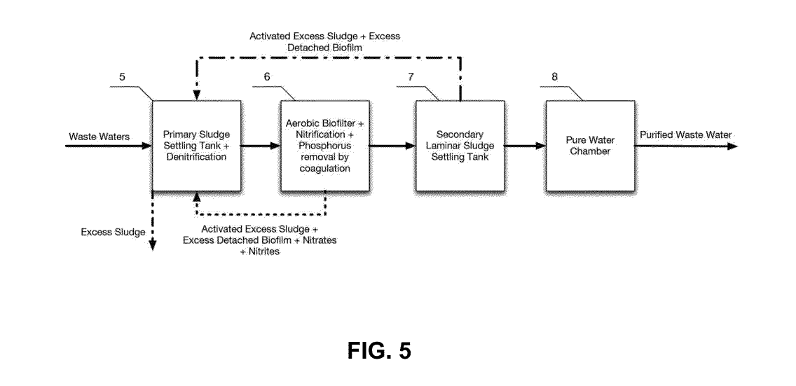

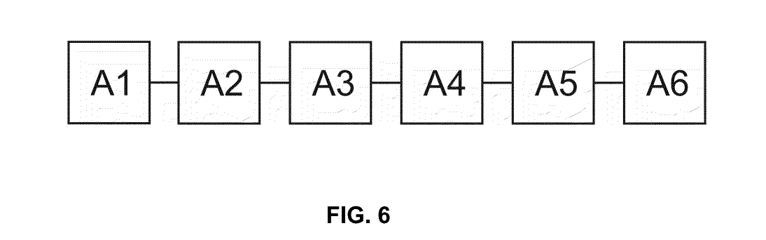

As shown in FIGS. 5 and 6, the plant has a Primary Sludge Settling Tank 5, a Aerotank Bio-filter 6, a Secondary Sludge Settling Tank 7 and a Pure Water Chamber 8 that are connected in-line.

Step A1. The biological load for bioreactors of the present invention are placed in the Aerotank Bio-filter 6, specifically in the biological filter of the bioreactor.

Step A2. Colonies of microorganisms, which are the basis of the bio-filter, begin to multiply and colonize the space formed by the loops and canvas of the biological load for bioreactors.

Step A3. Wastewater is fed into the first chamber 5 which acts as a primary sludge settling tank 5 and a receiver-accumulator operating as a system recycling activated sludge and nitrified substances in the chamber 7. Nitrates and nitrites received from the wastewater chamber 7 by the recirculation system are anaerobically biodegraded by microorganisms into oxygen, which they need for life, and nitrogen, which is released as a gas.

Step A4. Next, the wastewater flows for purification into bioreactor 6, which in this case, is both an aeration tank and a bio-filter. In this tank, microorganism biocenoses successfully exist in several states:
immobilized on submerged loading material biomass,
detached free-floating microorganisms.

Step A5. The bioreactor 6 is supplied with air, and the microorganisms begin to oxidize organic substances in the wastewater, thereby producing the energy they need to continue their lifecycle and also synthesizing their biomass. When all the organics are digested by the microorganisms, autotrophs start working instead of the heterotrophs, nitrifying the wastewater ammonia nitrogen to nitrites and nitrates in the aerobic conditions of the bioreactor.

Step A6. If repair, diagnostics, or replacement of bioreactor elements with the biological load for bioreactors is necessary, it is sufficient to pull out the desired element of the biological load out of the bioreactor.

### INDUSTRIAL APPLICABILITY

The presented biological load for bioreactors has a clear purpose, can be implemented by a specialist in practice, and the implementation ensures realization of the claimed purpose.

The prototype biological load for bioreactors was manufactured by an automated rotary thermoforming and mechanical shuttle hook-spoke-binding.

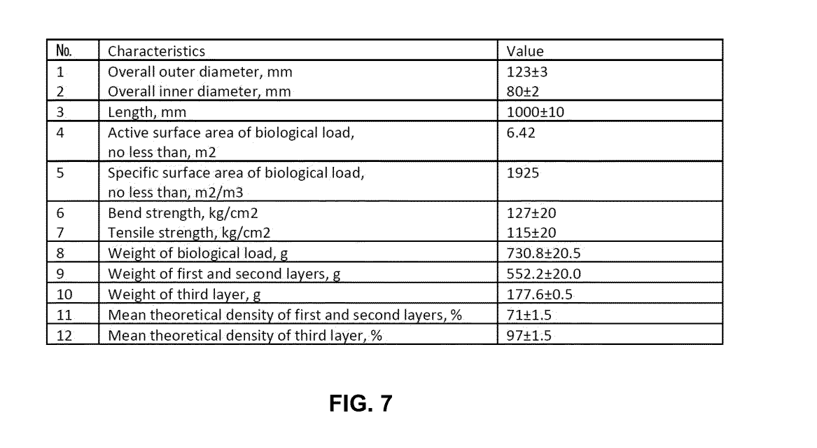

The biological load for bioreactors is a composite resin pipe with specified functional three-layer structure made by an optimized process of thermoforming and knitting, with geometric, physical and mechanical characteristics listed in the table in FIG. 7.

Trial operation of the utility model has shown that the design of the biological load for bioreactors provides:
improved efficiency of the biofilter due to rapid biomass growth,
reduced overall dimensions of treatment facilities.

Thus, by making the biological load for bioreactors consisting of three layers with the above parameters, the technical result is achieved, namely the improved technical specifications of the biological load.
improved ratio between the surface area and the occupied volume of the biological load,
increased corrosion resistance,
increased structural integrity of the frame of the biological load,
improved practicability of the assembly and maintenance of the biological load.

Thus, it is recommended to use the proposed biological load for bioreactors for installation, both in flow aeration tanks and in aeration tanks of variable action, in stations for deep biochemical and biological treatment of wastewater from residential complexes, hotels, guesthouses, resorts, building complexes, cottage communities, neighborhoods, towns, etc.

The foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

With respect to the above description, it is to be realized that the optimum relationships for the parts of the invention in regard to size, shape, form, materials, function and manner of operation, assembly and use are deemed readily apparent and obvious to those skilled in the art, and all equivalent relationships to those illustrated in the drawings and described in the specification are intended to be encompassed by the present invention.

What is claimed is:

1. A biological load for a bioreactor having a cylindrical shaped housing with an inner surface and an outer surface, said housing comprises of,
a. an inner layer having a diameter is placed at said inner surface made of a low-density polyethylene with a polymorphic structure;
b. a middle layer to form a supporting frame of the biological load having a gradient porous transition into an outer layer;
c. said outer layer formed by a canvas of polymer filaments made of polypropylene fiber, said canvas formed as a plurality of rowlock arch shaped distributed over said outer surface.

2. The biological load for a bioreactor of claim 1, wherein said inner layer having a porosity of up to 71%.

3. The biological load for a bioreactor of claim 1, wherein said inner layer further having a linear value for the size of the pores up to 1.5 mm.

4. The biological load for a bioreactor of claim 1, wherein the surface area of the space between the inner and outer layers are at least 750 m²/m³.

5. The biological load for a bioreactor of claim 1, wherein said middle layer having a diagonal thickness ranging from 0.5 to 2% of the diameter of the inner layer.

6. The biological load for a bioreactor of claim 1, wherein said middle layer having a gradient porous transition into said outer layer.

7. The biological load for a bioreactor of claim 1, wherein said middle layer having a ratio between the sizes of the pore channels of the inner and the outer layers equal to two.

8. The biological load for a bioreactor of claim 1, wherein said outer layer having a specific surface area of at least 1220 m²/m³.

9. The biological load for a bioreactor of claim 1, wherein said outer layer having a density of at least 95%.
